(19) United States
(12) Patent Application Publication
(10) Pub. No.: US 2025/0242074 A1
(43) Pub. Date: Jul. 31, 2025

(54) Scent Diffusion Using Burst Dispersion in a Scent Dispenser

(71) Applicant: Pura Scents, Inc., Pleasant Grove, UT (US)

(72) Inventors: Trevor Davis, American Fork, UT (US); Auston Tesch, Pleasant Grove, UT (US)

(21) Appl. No.: 19/043,149

(22) Filed: Jan. 31, 2025

Related U.S. Application Data
(60) Provisional application No. 63/627,351, filed on Jan. 31, 2024.

(51) Int. Cl. A61L 9/03

(52) U.S. Cl. CPC A61L 9/035; A61L 2209/11; A61L 2209/133; A61L 2209/135

(57) ABSTRACT

A method of emitting a scent from a scent dispensing device, comprising determining an identity of a scent in a scient vial positioned within a scent dispensing device, determining a spread trait of the scent in the scent vial based on the identity, determining a scent strength setting of the scent dispensing device, determining a burst diffusion setting based on the scent strength setting and the spread trait of the scent in the scent vial, and causing a dispensing mechanism of the scent dispensing device to begin emitting a scent in one or more bursts based on the burst diffusion setting.

### BACKGROUND

The present disclosure relates to scent diffusion using burst dispersion in a scent dispenser.

Existing solutions for dispensing scents within a room include liquid scented oil diffusers, wax or cork disks infused with scented oils, scent beads, or nebulizers. The scented oil can be dispersed throughout the room by heating elements and/or fans. To emit the scents in the rooms, the heating elements and/or fans are turned on to emit a scent and turned off to stop scent diffusion. Additionally, in some existing solutions, the intensity settings of the scent may be changed by increasing the temperature of the heating element and/or the speed of the fan to increase or decrease the amount of scent that is being emitted into the room.

### SUMMARY

In some aspects, the techniques described herein relate to a method of emitting a scent from a scent dispensing device, including: determining an identity of a scent in a scent vial positioned within a scent dispensing device; determining a spread trait of the scent in the scent vial based on the identity; determining a scent strength setting of the scent dispensing device; determining a burst diffusion setting based on the scent strength setting and the spread trait of the scent in the scent vial; and causing a dispensing mechanism of the scent dispensing device to begin emitting the scent in one or more bursts based on the burst diffusion setting.

In some aspects, the techniques described herein relate to a method, wherein the burst diffusion setting includes a first burst period of a plurality of burst periods in which a scent is emitted as a first burst of the one or more bursts and a delay period before a subsequent burst period.

In some aspects, the techniques described herein relate to a method, wherein the delay period is periodic and allows for even burst periods from the plurality of burst periods.

In some aspects, the techniques described herein relate to a method, wherein the delay period is random and allows for uneven burst periods from the plurality of burst periods.

In some aspects, the techniques described herein relate to a method, wherein the delay period includes designated intervals based on reducing nose blindness.

In some aspects, the techniques described herein relate to a method, wherein the designated intervals include a first designated interval and a second designated interval that are different.

In some aspects, the techniques described herein relate to a method, wherein the spread trait is a characteristic of how scent molecules of the scent spread through an area.

In some aspects, the techniques described herein relate to a method, further including: determining a temperature of the scent as the scent is emitted; updating the spread trait based on the temperature; and adjusting the burst diffusion setting based on the temperature and the updated spread trait.

In some aspects, the techniques described herein relate to a method, wherein different scents have different spread traits at different temperatures.

In some aspects, the techniques described herein relate to a scent dispensing device including: a body including a cavity in which one or more scent vials may be positioned; and a dispensing element configured to cause the one or more scents to emit a scent by: determining an identity of one or more scents in the one or more scent vials positioned within a scent dispensing device; determining spread traits of the one or more scents in the one or more scent vials based on the identities; determining scent strength settings of the one or more scents in the one or more scent vials; determining burst diffusion settings based on the scent strength settings and the spread traits of the one or more scents in the one or more scent vials; and causing a dispensing mechanism of the scent dispensing device to begin emitting one or more scents in the one or more scent vials in one or more bursts based on the burst diffusion setting.

In some aspects, the techniques described herein relate to a scent dispensing device, wherein the burst diffusion settings for each one or more scents includes a first burst period of a plurality of burst periods in which each scent of the one or more scents is emitted as a first burst of the one or more bursts and a delay period before a subsequent burst period.

In some aspects, the techniques described herein relate to a scent dispensing device, wherein the delay period is periodic and allows for even burst periods from the plurality of burst periods.

In some aspects, the techniques described herein relate to a scent dispensing device, wherein the delay period is random and allows for uneven burst periods from the plurality of burst periods.

In some aspects, the techniques described herein relate to a scent dispensing device, wherein the delay period includes designated intervals based on reducing nose blindness.

In some aspects, the techniques described herein relate to a scent dispensing device, wherein the designated intervals include a first designated interval and a second designated interval that are different.

In some aspects, the techniques described herein relate to a scent dispensing device, wherein the spread trait is a characteristic of how scent molecules of the scent spread through an area.

In some aspects, the techniques described herein relate to a scent dispensing device, wherein the dispensing element includes a plurality of heating elements for each of the one or more scent vials, and wherein each scent is emitted from the one or more scent vials as each of the heating elements of the plurality of heating elements causes each of the scent vials of the plurality of scent vials to increase in temperature, scents are emitted.

In some aspects, the techniques described herein relate to a scent dispensing device, further including: determining temperatures of each the heating elements as each scent is emitted; updating the spread trait based on the temperature; and adjusting the burst diffusion setting based on the temperature and the updated spread trait.

In some aspects, the techniques described herein relate to a method of emitting a scent from a scent dispensing device, including: determining a first identity of a first scent in a first scent vial positioned within a scent dispensing device; determining a second identity of a second scent in a second scent vial positioned within a scent dispensing device; determining a first burst diffusion setting for the first scent in the first scent vial based on the first identity; determining a second burst diffusion setting for the second scent in the second scent vial based on the second identity; and causing a dispensing mechanism of the scent dispensing device to begin emitting one or more of the first scent and the second scent in one or more bursts based on the first burst diffusion setting and the second burst diffusion setting.

In some aspects, the techniques described herein relate to a method, wherein the first burst diffusion setting for the first scent in the first scent vial includes a spread trait of the first scent and the second burst diffusion setting for the second scent in the second scent vial includes a spread trait of the second scent.

However, this list of features and advantages is not all-inclusive, and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1 is a block diagram illustrating an example system for scent diffusion using burst dispersion in a scent dispenser.

FIGS. 2A and 2B depict a graphical representation of a scent dispensing device emitting a burst scent pattern in a space.

FIG. 3A and 3B depict a graphical representation of a scent dispensing device emitting a burst scent pattern in a vehicle.

FIG. 4 depicts a graphical representation of multiple scent dispensing devices emitting burst patterns in a space.

FIG. 5 is a flowchart of an example method of scent diffusion using burst dispersion in a scent dispenser.

### DETAILED DESCRIPTION

The technology described in this disclosure relates to scent diffusion using burst dispersion in a scent dispenser. As an example, the technology allows for a scent dispenser to dispense a scent for a duration, referred to as a “burst,” and then stop emitting the scent for a duration before emitting another burst for another period of time. This burst dispersion effect, where a concentrated amount of scent is released for a period of time and then the scent dispenser waits for a period of time before releasing another amount of the scent for a period of time, such as a few seconds allows, for greater spread of the scent throughout an area, such as a room or car interior, and also reduces nose blindness from a user, where the user gets accustomed to the scent being dispersed and no longer notices it because there is no change in the scent over time.

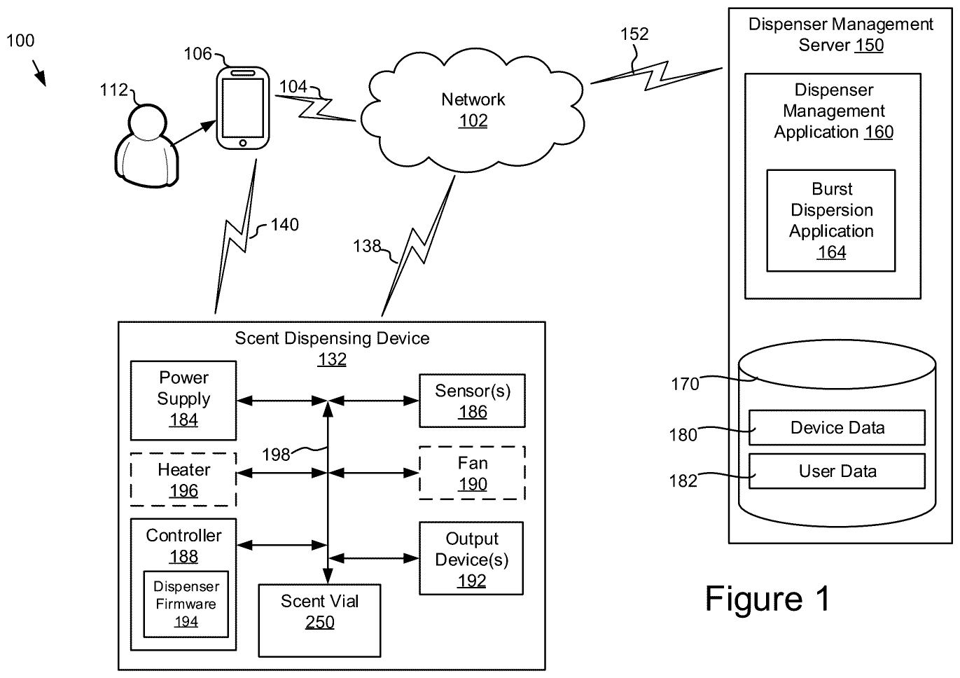

FIG. 1 is a block diagram illustrating an example system 100 for scent diffusion using burst dispersion in a scent dispenser. The system 100 may include one or more scent dispensing devices 132 positioned in an area, such as a room, space, vehicle, etc. In a typical implementation, a single scent dispensing device 132 is positioned within a space, however in large spaces multiple scent dispensing devices 132 may be positioned throughout the space for effective scent dispensing where the devices 132 may work together and in some implementations may adapt different burst dispersion patterns in order to fully disperse a scent through a space. In some implementations, the scent dispensing device 132 may plug into a wall outlet to provide power to the scent dispensing device 132. In other implementations, the scent dispensing device 132 may be battery powered, such as a car device or other portable device that is positioned within a vehicle or other space. In some implementations, the scent dispensing device 132 may include a body with a cavity or space in which one or more scent vials 250 may be positioned. In some implementations, the one or more scent vials 250 may be positioned adjacent to one or more dispensing elements, such as a heating element for a liquid scent or a fan to cause airflow over a solid scent, such as a gel or bead.

As shown, the illustrated system 100 further includes client device(s) 106 and a server 150, which are electronically communicatively coupled via a network 102 for interaction with one another and the scent dispenser(s) 132, etc., using standard networking protocols, as reflected by signal lines 104, 138, and 152. In some implementations, the scent dispensing device 132 may instead by communicatively coupled to the client device(s) 106, such as by Bluetooth, beacons, or other networking protocols (as reflected by signal line 140) in order to allow the scent dispensing device 132 to connect with a paired client device(s) 106 when in proximity rather than through the network 102.

The dispenser management application 160 operable by the dispenser management server 150 can receive operational data from the scent dispensing device 132 in association with the device 106 and/or user(s) 112 with which they are associated. The dispenser management application can receive management requests for a dispensing device 132, such as a diffusion level, burst dispersion patterns, spread traits of various scents, scent choice, or various data that can be analyzed over time to improve the scent dispensing experience for the user 112.

The dispenser management application 160 can include a burst dispersion application 164. The burst dispersion application 164 may include software and/or logic to scent diffusion using burst dispersion in a scent dispenser. The burst dispersion application 164 can provide setting information to the one or more scent dispensing devices 132 to allow a dispensing mechanism to output one or more burst patterns for scent diffusion.

The dispenser management server 150 includes a data store 170 storing various types of data used by the dispenser management application 160. Example data types include device data 180 and user data 182. The device data 180 may include a device model, a scent vial type, usage statistics, scent diffusion time, scent spread traits, temperature variations, etc. The user data 182 may include entries for the users 112 of the system 100. A given entry may include a unique identifier for the user, a unique identifier for the user device 106, contact information for the user (e.g., address, phone number, electronic address (e.g., email)), payment information, scent subscription information specifying which reoccurring scent vials 250 should be shipped to the user, etc.

An example scent dispenser device 132 is depicted as including a power supply 184, one or more sensor(s) 186, a controller 188, output device(s) 192, dispenser firmware 194, a fan 190, a heater 196, and any number of scent vial(s) 250. The components 184, 186, 188, 190, 192, 196, and 250 are communicatively coupled via a communications bus 198. The controller 188 may include a non-transitory memory device (e.g., a non-volatile memory device), or may be coupled to a non-transitory memory device also coupled for communication via the bus 198. The non-transitory memory device may store software and/or firmware that specially configures the controller, such as the dispenser firmware 194. The power supply 184 may be any AC and/or DC power supply for powering the scent dispensing device 132. In some implementations, the power supply 184 may be battery powered and may be configured to charge when plugged into an AC and/or DC power supply or positioned on a wireless charging base. The controller 188 may be a microchip that controls the constituent electronics (e.g., sensor(s) 186, output device(s) 192, fan 190, heater 196, etc.) of the scent dispensing device 132.

The one or more sensor(s) 186 may include one or more temperature sensors for detecting the ambient temperature adjacent to the scent dispensing device 132 and can use those readings to determine efficient diffusion of the scent along with the dynamic scent settings from the dynamic scent application 164. In some implementations, the one or more sensors(s) 186 may include a humidity to determine efficient diffusion of the scent based on various humidity readings. In some implementations, the one or more sensor(s) 186 may include a vial sensor for sensing when to replace a scent vial(s) 250 installed in the scent dispensing device 132, optical or other sensor(s) or electronics for detecting an identity of scent vial(s) 250 installed in the scent dispensing device 132, ambient light sensor to detect a light level in a surrounding environment, and/or a motion sensor to detect motion in the surrounding environment, etc. In some embodiments, the sensors may include an audio sensor to detect audio sounds in the surrounding environment. In some embodiments, the sensors 186 may include a separate temperature sensor for the scent vial 250 (e.g., to measure temperatures at the scent vial 250, which can be used to adjust a speed and/or frequency of the fan 190).

The sensors 186 may include a transceiver having a wireless interface configured to communicate with the devices coupled to the network 102, such as the dispenser management server 150, and/or other components of the network 102 using standard communication protocols, such as Internet protocols. Further, the transceiver may be configured to wirelessly transmit data via a network to connect to other devices, such as the mobile device 106. By way of further example, the transceiver may transmit data to the mobile device 106 to which it is linked using a protocol compliant with IEEE 802.15, such as Zigbee®, Z-Wave®, Bluetooth®, or another suitable standard. Further embodiments are also possible and contemplated. In some embodiments, the transceiver may be embedded in the controller 188 or may be a component distinct from the controller and coupled to the controller 188 via the bus 198.

The output device(s) 192 may include light sources and/or audio reproduction devices, although further suitable output devices are also contemplated and applicable. In some implementations, the light sources and/or audio reproduction devices may be controlled to produce output consistent with a scent being emitted by the scent dispenser (e.g., a low, soothing light and music may be output in conjunction with a relaxing scent being emitted), or to communicate various alerts, such as low power, low scent vial levels, etc. In some implementations, the output device(s) 192 may correlate a sent dispensing output with a burst output where a light flashes or a low hum is released when a burst of scent is emitted.

In some implementations, the scent dispensing device 132 may include the fan 190. The fan 190 may include a motor that has one or more fan blades that force air through the device 132 when the motor is operating. The fan 190 may operate at various speeds based on how quickly or slowly the motor runs. The fan 190 may be configured to nest within a housing of the device 132 and cause airflow to move through the device and across a scent vial 250 for scent diffusion. In some implementations, the fan 190 may be able to turn on and off as signaled by the microcontroller 188 which results in substantially immediate scent diffusion. In some implementations the fan 190 and the motor speed may be associated with scent settings and based on the level of the motor operating the fan 190 different scent diffusions can be released to fill different areas over different times based on the scent settings. In some implementations for burst settings, the fan 190 may operate only when a burst is activate to flow over a scent vial 250 and emit a scent. In other implementations, the fan 190 may be continuously operating and a scent release mechanism may open and/or close when bursts are on in order to allow a burst of scent to be emitted.

The scent vial 250 may be removable and contain a liquid scenting agent that diffuses a scent into the nearby air. The scent vial 250 may be replaceable when the scent is diminished and a new scent vial 250 can be inserted into the device 132. The scent vials 250 may have various scent profiles and information about the scent vials 250 may be stored in the dispenser management application 160 to store the various scent profiles, ages of the scent vials 250, duration of use of the scent vials 250, exposed temperatures of the scent vials 250, etc.

The client device(s) 106 (also referred to individually and collectively as 106) are computing devices having data processing and communication capabilities. In some embodiments, a client device 106 may include a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The client devices 106 may couple to and communicate with one another and the other entities of the system 100 via the network 102 using a wireless and/or wired connection. Examples of client devices 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, car access panels, etc. In addition, while a single client device 106 is depicted in FIG. 1, it should be understood that any number of client devices 106 may be included.

As shown, the client device 106 may include a scent application, which allows the user to set scent dispensing device 132 settings, turn scent dispensers 132 on and off, purchase scent vials 250 for the scent dispensing device 132, provide feedback about optimized scent settings, set up a scent dispensing device 132, register an account, view analytics reflecting the user's historical use of his/her scent dispensing device 132, enable user profiles to use and setup scent profiles for the scent dispensing device 132, set a profile hierarchy (e.g., set which user profile(s) is/are the dominant user profile), set motor speeds for the fan 190, manage various scent settings, etc.

The dispenser management server 150 may include one or more computing devices having data processing, storing, and communication capabilities. For example, the server 150 may include one or more hardware servers, virtual servers, server arrays, storage devices and/or systems, etc., and/or may be centralized or distributed/cloud-based. In some embodiments, the server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the server 150 may include a (physical, virtual, etc.) processor, a non-transitory memory, a network interface, and a data store 170, which may be communicatively coupled by a communications bus. Similarly, the client device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 150 (e.g., the dispenser management application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 150 may include a dispenser management application 160 embodying a remotely accessible scent service. The dispenser management application 160 may send data to and receive data from the other entities of the system including the controllers 188, the mobile device(s) 106, etc. The dispenser management application 160 may be configured to store and retrieve data from one or more information sources, such as the data store 170. In addition, while a single server 150 is depicted in FIG. 1, it should be understood that one or more servers 150 may be included.

In some embodiments, the dispenser firmware 194, the scent application, the dispenser management application 160, the burst dispersion application 164, etc., may require users to be registered to access the acts and/or functionality provided by them. For example, to access various acts and/or functionality provided by the scent application, dispenser management application 160, and/or scent dispensers 132, these components may require a user to authenticate his/her identity (e.g., by confirming a valid electronic address). In some instances, these entities 132, 150, etc., may interact with a federated identity server (not shown) to register/authenticate users. Once registered, these entities 132, 150, etc., may require a user seeking access to authenticate by inputting credentials in an associated user interface.

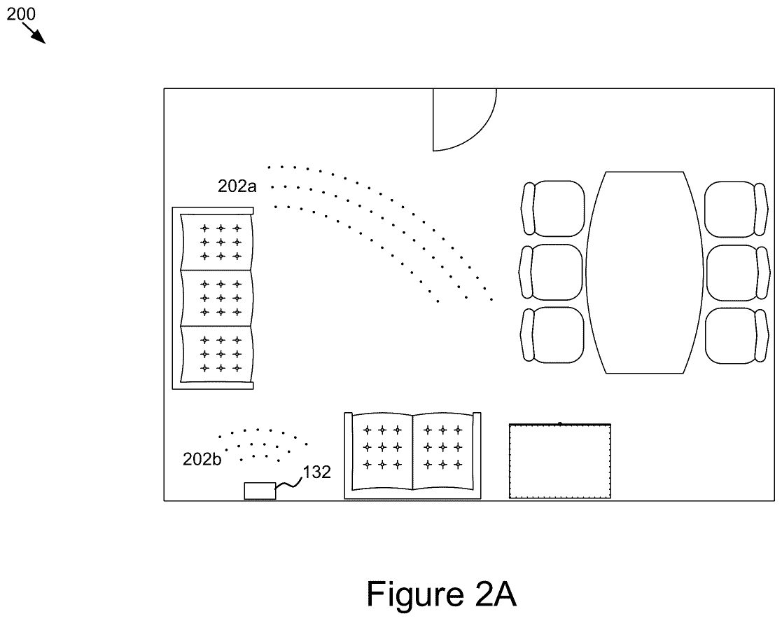

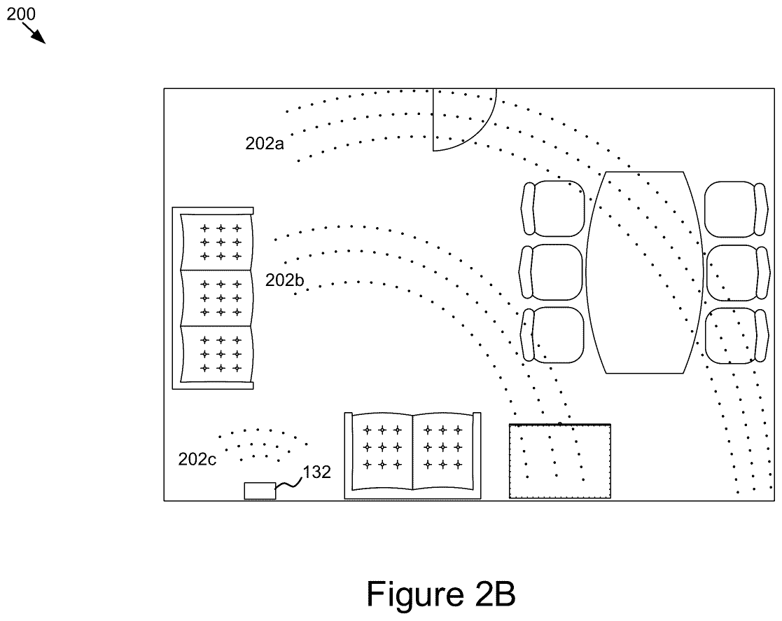

FIGS. 2A and 2B depict a graphical representation of a scent dispensing device 132 emitting a burst scent pattern in a space 200. As shown in FIG. 2A, a scent dispensing device 132 may be positioned in a space, such as a room with furniture or other objects in the space. When the scent dispensing device 132 is activated and a burst dispersion setting begins dispersing a scent, an amount of scent 202 is released from the scent dispensing device. For example, as shown in FIG. 2A, a burst setting is selected that emits three waves of scent 202a then stops emitting scent for a period of time before emitting a second burst of three waves of scent 202b as shown. Once the scent particles have been emitted, they spread through the space to begin diffusing the scent. Because of the way the burst pattern releases the scent, the first burst may be somewhat clustered together and spread out more over time as shown in FIG. 2A. It should be understood that the three wave burst pattern shown in FIGS. 2A and 2B is merely illustrative to depict scent being dispersed in a space and other burst patterns and examples are also contemplated. In some implementations, based on where the scent dispensing device 132 is positioned within a space, such as along a far wall, or behind some furniture, etc. it may have to adjust a burst pattern in order to efficiently spread a scent throughout the space. For example, a first burst may be stronger to allow a strong initial scent dispersion and then later bursts may be less than the first in order to maintain the scent without overwhelming the initial scent. In other implementations, the burst pattern may be focused on creating gaps or spaces within the area between burst waves where the scent cannot be detected in order to reduce nose blindness in a user 112 where the bursts become noticeable because they are different from the ambient scent of the space, etc.

As shown in FIG. 2B, after a period of time, a third burst of three waves of scent 202c may be emitted from the scent dispensing device 132 based on the burst pattern. As shown in FIG. 2B, the previous waves of scent 202a and 202b may continue to spread throughout the space as the third burst of three waves of scent 202c is emitted. In some implementations, the burst dispersion application 164 may have various learning modules or artificial intelligence that can learn over time how many bursts should be released to fill a room with a scent to a desired scent intensity (e.g., a low scent intensity, a medium scent intensity, and/or a high scent intensity).

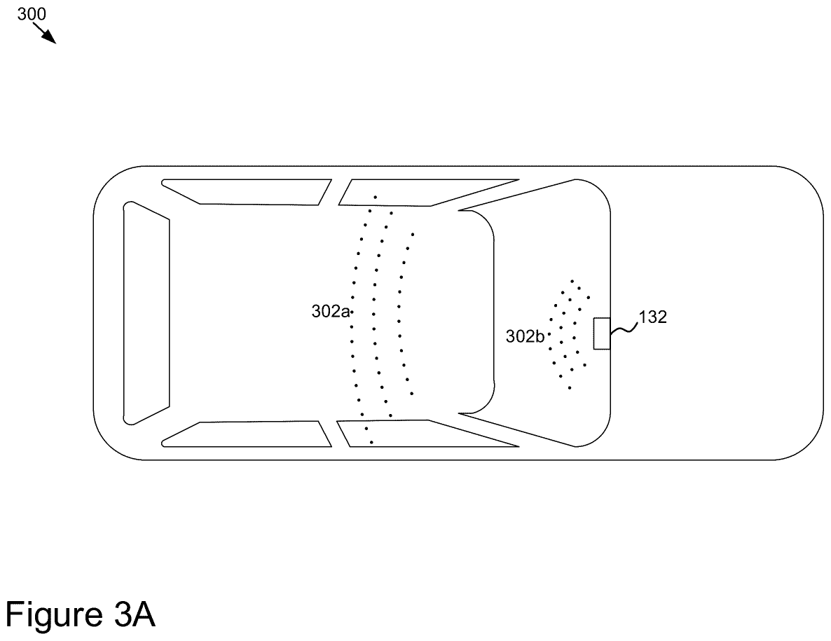

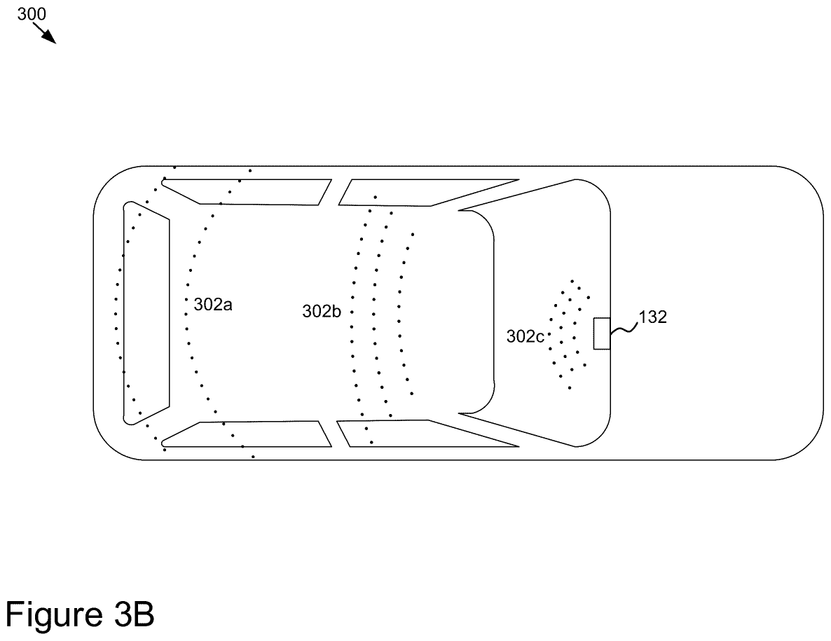

FIG. 3A and 3B depict a graphical representation of a scent dispensing device 132 emitting a burst scent pattern in a vehicle 300. In some implementations, as shown in FIG. 3A, a vehicle, such as a car, bus, train, airplane, etc. may have a scent dispensing device 132 positioned within to dispense scent. Generally, a vehicle has a smaller space and/or different scent needs as compared to a room, where in a vehicle each area in the vehicle needs more of a personal space scent and in a room, the scent is dispersed throughout the room area. As shown in FIG. 3A, a wave dispersion pattern 302 to emit the scent in the vehicle. A first wave dispersion pattern 302a was emitted and then after a period of time, a second wave dispersion pattern 302b was emitted as shown. The bursts of scent then move through the space in the interior of the vehicle to disperse the scent through the vehicle. FIG. 3B depicts a third wave dispersion pattern 302c being emitted in a burst pattern from the scent dispensing device 132. Furthermore, in some implementations, as shown, the earlier scent waves may begin to die off as shown with the spread out and reduced number of scent particles in the first wave dispersion pattern 302a. The burst dispersion application 164 can account for scent die off as the scent is spread throughout the space. In one example, a burst dispersion on a low setting may be 20 seconds of scent dispersion followed by 30 seconds where the device is not dispersing scent, followed by another 20 seconds of scent dispersion and another 30 seconds of not dispersing scent. This is just an example, and other scent time periods for burst emission are also contemplated in different patterns and/or at different intensities. Furthermore, during the burst emission, the heating elements 196 and/or the fan 190 may also have different intensity settings to change the burst dispersion pattern intensity, etc.

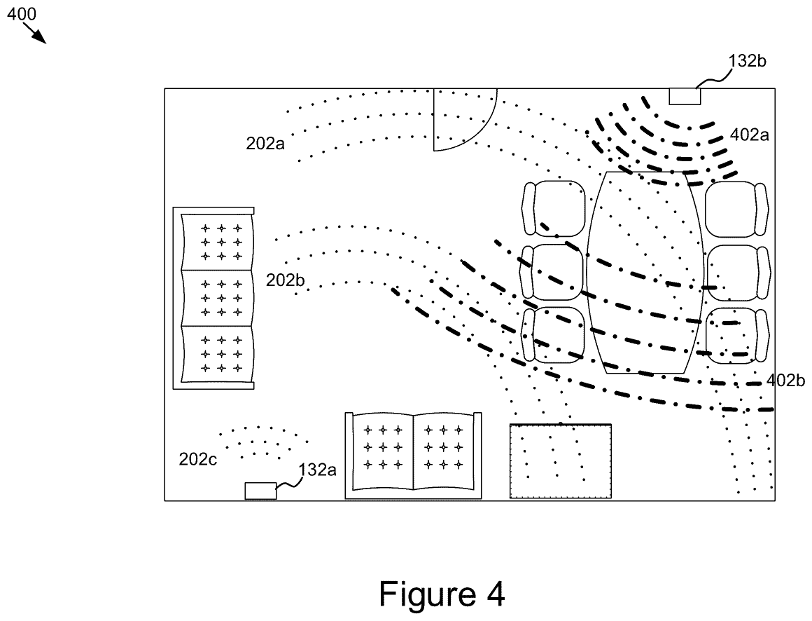

FIG. 4 depicts a graphical representation of multiple scent dispensing devices 132 emitting burst patterns in a space 400. As shown, a first scent dispensing device 132a may be emitting a first type of burst patterns as shown by scent bursts 202a-202c. Furthermore, a second scent dispensing device 132b may be emitting a second type of burst patterns as shown by scent bursts 402a-402b. In some implementations both the first scent dispensing device 132a and the second scent dispensing device 132b may be emitting a same or complementary scent from the scent vials 250, while in other implementations, the scents may not be the same or complementary, such as to cancel scents out, etc. As shown, the two scent dispensing devices 132a and 132b are used to more quickly and efficiently fill the room with a scent. In addition, the two scent dispensing devices 132a and 132b may be communicating with each other in order to coordinate burst dispersions of scent to achieve a goal. For example, an area of a space, such as a conference room table, may experience higher user traffic and thus may benefit from a more concentrated scent dispersion bursts in only that area. By using the two scent dispensing devices 132a and 132b with a same/similar or different type of burst settings, the high traffic area can benefit from a more concentrated scent diffusion, while the rest of the room may receive a lighter scent diffusion based on the burst settings. In those situations, positions of the scent dispensing devices 132 and layout of the room may be used along with artificial intelligence and/or machine learning to determine the optimal burst settings for each scent dispensing device 132.

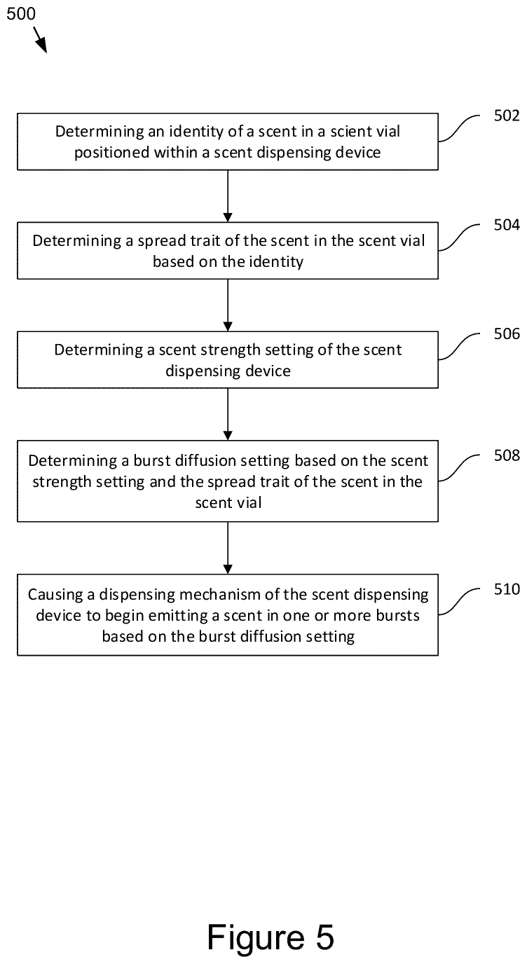

FIG. 5 is a flowchart of an example method 500 of scent diffusion using burst dispersion in a scent dispenser. At 502, a burst dispersion application 164 can determine an identity of a scent in a scent vial 250 positioned within a scent dispensing device 132. In some implementations, different scents may have different characteristics and determining the identity may assist the burst dispersion application 164 in determining optimal burst settings. The identify of the scent may be determined by identifying a label or smart label detectable by a sensor 186, or a user 112 may manually input or store the scent identity when a scent vial 250 is installed etc.

At 504, the burst dispersion application 164 can determine a spread trait of the scent in the scent vial 250 based on the identity. The spread trait may be a characteristic of how a scent molecule moves through an area at different temperature levels and/or concentrations, as well as how a scent life of the scent molecule may change over time. Using the spread trait, the burst dispersion application 164 can determine how that exact scent would act in a space at different temperature levels and/or fan 190 settings, etc.

At 506, the burst dispersion application 164 may determine a scent strength setting of the scent dispensing device 132. The scent strength setting may be a user 112 setting, such as a low scent intensity, a medium scent intensity, and/or a high scent intensity that a user 112 has set for a space or vehicle, etc. In some implementations, the scent strength setting may be automatically set by the burst dispersion application 164 based on information about a room, such as size and/or airflow rate, or user preferences from previous scent settings.

At 508, the burst dispersion application 164 can determine a burst diffusion setting based on the scent strength setting and/or the spread trait of the scent in the scent vial. The burst diffusion setting may be optimally determined by the burst dispersion application 164 based on a size of the space and how strong the scent would need to be and spread within the size of the space. Additionally, different burst diffusion settings may benefit from different spread traits and/or scent strength settings and the burst dispersion application 164 can factor on those two elements to determine the optimal burst diffusion settings. In some implementations, the burst diffusion setting may include burst periods, where during a burst period a scent is emitted in a burst from the scent dispensing device following by a delay period in which no scent is emitted, followed by an additional or subsequent burst period in which another burst is emitted from the scent dispensing device. These burst periods may be used to combat nose blindness of one or more users, where the scent is emitted during the burst period and then the scent emission is halted during the delay period and then a subsequent burst period causes a user to detect the smell again as compared to a scent that is constantly being dispersed. In some implementations, the delay period and/or the burst periods may be have period intervals and the burst pattern may be an even burst pattern. In further implementations, the delay period and/or the burst periods may be uneven, such as random or slightly random (where the interval has a preset period plus or minus an additional random period) in order to further combat nose blindness by randomly having bursts being emitted based on the burst diffusion settings. In further implementations, the delay period and/or the burst periods may have designated intervals. In some implementations, these designated intervals may be based on nose blindness data, such as if the nose blindness being avoided is short term, medium, term, or long term. In further implementations, the designated intervals may be preset by a user to change the burst diffusion settings. In some implementations, the designated intervals may be different for different users or different scents in the scent dispensing device. In some implementations, the designated intervals may be different within a scent emitting period, such as having a first designated interval and a second designated interval.

At 510, the burst dispersion application 164 can cause a dispensing mechanism, such as a heater 196 and/or fan 190, of the scent dispensing device 132 to begin emitting a scent in one or more bursts based on the burst diffusion setting. The bursts may be scent outputs for a period of time, followed by no scent output for a period of time, followed by another scent output for a period of time to create a burst pattern of scent emission followed by no scent emission. These burst patterns can follow all sorts of different pattern outputs and are not limited to the examples described elsewhere herein. In some implementations, the burst may cause the scent to be dispersed throughout the space efficiently without requiring the scent dispensing device 132 to be on continually during that duration. This may improve scent diffusion, while also increasing a lifespan of the scent since it's not being continually output. This may also increase enjoyment and or engagement with a user 112 as they will not become nose blind to a scent when it is emitted in bursts with gaps between the bursts. In some implementations, the temperature of the heating element or dispensing element may be monitored and the burst diffusion setting may be changed as the temperature of the heating elements may be changed. For example, as a temperature may change for the scent adjacent to the heating element, the spread trait may increase or decrease and the burst diffusion settings may be changed based the change or update in the spread trait.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A method of emitting a scent from a scent dispensing device, comprising:
determining an identity of a scent in a scent vial positioned within a scent dispensing device;
determining a spread trait of the scent in the scent vial based on the identity;
determining a scent strength setting of the scent dispensing device;
determining a burst diffusion setting based on the scent strength setting and the spread trait of the scent in the scent vial; and
causing a dispensing mechanism of the scent dispensing device to begin emitting the scent in one or more bursts based on the burst diffusion setting.

2. The method of claim 1, wherein the burst diffusion setting includes a first burst period of a plurality of burst periods in which a scent is emitted as a first burst of the one or more bursts and a delay period before a subsequent burst period.

3. The method of claim 2, wherein the delay period is periodic and allows for even burst periods from the plurality of burst periods.

4. The method of claim 2, wherein the delay period is random and allows for uneven burst periods from the plurality of burst periods.

5. The method of claim 2, wherein the delay period includes designated intervals based on reducing nose blindness.

6. The method of claim 5, wherein the designated intervals include a first designated interval and a second designated interval that are different.

7. The method of claim 1, wherein the spread trait is a characteristic of how scent molecules of the scent spread through an area.

8. The method of claim 1, further comprising:
determining a temperature of the scent as the scent is emitted;
updating the spread trait based on the temperature; and
adjusting the burst diffusion setting based on the temperature and the updated spread trait.

9. The method of claim 8, wherein different scents have different spread traits at different temperatures.

10. A scent dispensing device comprising:
a body including a cavity in which one or more scent vials may be positioned; and
a dispensing element configured to cause the one or more scents to emit a scent by:
determining an identity of one or more scents in the one or more scent vials positioned within a scent dispensing device;
determining spread traits of the one or more scents in the one or more scent vials based on the identities;
determining scent strength settings of the one or more scents in the one or more scent vials;
determining burst diffusion settings based on the scent strength settings and the spread traits of the one or more scents in the one or more scent vials; and
causing a dispensing mechanism of the scent dispensing device to begin emitting one or more scents in the one or more scent vials in one or more bursts based on the burst diffusion setting.

11. The scent dispensing device of claim 10, wherein the burst diffusion settings for each one or more scents includes a first burst period of a plurality of burst periods in which each scent of the one or more scents is emitted as a first burst of the one or more bursts and a delay period before a subsequent burst period.

12. The scent dispensing device of claim 11, wherein the delay period is periodic and allows for even burst periods from the plurality of burst periods.

13. The scent dispensing device of claim 11, wherein the delay period is random and allows for uneven burst periods from the plurality of burst periods.

14. The scent dispensing device of claim 11, wherein the delay period includes designated intervals based on reducing nose blindness.

15. The scent dispensing device of claim 14, wherein the designated intervals include a first designated interval and a second designated interval that are different.

16. The scent dispensing device of claim 10, wherein the spread trait is a characteristic of how scent molecules of the scent spread through an area.

17. The scent dispensing device of claim 10, wherein the dispensing element includes a plurality of heating elements for each of the one or more scent vials, and wherein each scent is emitted from the one or more scent vials as each of the heating elements of the plurality of heating elements causes each of the scent vials of the plurality of scent vials to increase in temperature, scents are emitted.

18. The scent dispensing device of claim 17, further comprising:
determining temperatures of each the heating elements as each scent is emitted;
updating the spread trait based on the temperature; and
adjusting the burst diffusion setting based on the temperature and the updated spread trait.

19. A method of emitting a scent from a scent dispensing device, comprising:
determining a first identity of a first scent in a first scent vial positioned within a scent dispensing device;
determining a second identity of a second scent in a second scent vial positioned within a scent dispensing device;
determining a first burst diffusion setting for the first scent in the first scent vial based on the first identity;
determining a second burst diffusion setting for the second scent in the second scent vial based on the second identity; and
causing a dispensing mechanism of the scent dispensing device to begin emitting one or more of the first scent and the second scent in one or more bursts based on the first burst diffusion setting and the second burst diffusion setting.

20. The method of claim 19, wherein the first burst diffusion setting for the first scent in the first scent vial includes a spread trait of the first scent and the second burst diffusion setting for the second scent in the second scent vial includes a spread trait of the second scent.
